(19) United States
(12) Patent Application Publication
(10) Pub. No.: US 2012/0194783 A1
(43) Pub. Date: Aug. 2, 2012

(54) COMPUTER-AIDED DIAGNOSIS OF RETINAL PATHOLOGIES USING FRONTAL EN-FACE VIEWS OF OPTICAL COHERENCE TOMOGRAPHY

(75) Inventors: Jay Wei, Fremont, CA (US); Bruno Lumbroso, Rome (IT); Ben Jang, Cupertino, CA (US); John Davis, San Jose, CA (US)

(73) Assignee: Optovue, Inc., Fremont, CA (US)

(21) Appl. No.: 13/360,503

(22) Filed: Jan. 27, 2012

Related U.S. Application Data
(60) Provisional application No. 61/437,449, filed on Jan. 28, 2011.

(51) Int. Cl. A61B 3/14

(52) U.S. Cl. 351/206; 351/246

(57) ABSTRACT

A system and methods of computer-aided diagnosis for ophthalmology are described that includes acquiring OCT data, determining an RPE fit from the OCT data, and displaying en face images based on the RPE fit.

### RELATED APPLICATIONS

This application claims priority to U.S. Provisional Application No. 61/437,449, filed on Jan. 28, 2011, which is herein incorporated by reference in its entirety.

### BACKGROUND

1. Field of the Invention

The embodiments described herein relate generally to methods and systems for processing and representing images in ophthalmology for diagnosis and treatment of diseases or any other physiological conditions.

2. Description of Related Art

Optical Coherence Tomography (OCT) is an optical signal and processing technique that captures three-dimensional (3D) data sets with micrometer resolution. This OCT imaging modality has been commonly used for non-invasive imaging of object of interest, such as retina of the human eye, over the past 15 years. A cross sectional retinal image as a result of an OCT scan allows users and clinicians to evaluate various kinds of ocular pathologies in the field of ophthalmology. However, due to limitation of scan speed in imaging device based on time-domain technology (TD-OCT), only a very limited number of cross-sectional images can be obtained for evaluation and examination of the entire retina.

A new generation of OCT technology, Fourier-Domain or Spectral Domain Optical Coherence Tomography (FD/SD-OCT), is significantly improved from TD-OCT, reducing many of the limitations of OCT such as data scan speed and resolution. 3D data set with dense raster scan or repeated cross-sectional scans can now be achieved by FD-OCT with a typical scan rate of approximately 17,000 to 40,000 A-scans per second. Newer generations of FD-OCT technology will likely further increase scan speed to 70,000 to 100,000 A-scans per second.

These technological advances in data collection systems are capable of generating massive amounts of data at an ever increasing rate. As a result of these developments, myriad scan patterns were employed to capture different areas of interest with different directions and orientations. A system and data presentation design is disclosed to more systematically present a 3D data set and to set a standard and consistent expectation of data representation for different clinical needs.

Current trends in ophthalmology make extensive use of 3D imaging and image processing techniques to generate high resolution images. Such images may be utilized for diagnosing diseases such as glaucoma, and other medical conditions affecting the human eye. One of the challenges posed by the current technological advances in imaging techniques is the efficient and meaningful processing and presentation of the massive amounts of data collected at ever increasing imaging rates. Some approaches have converted 3D data sets into manageable two-dimensional (2D) images to be analyzed. An example of such technique used for data reduction from a 3D data set to a 2D image is 2D “en-face” image processing. (See for example, Bajraszewski et al., [Proc. SPIE 5316, 226-232 (2004)], Wojtkowski et al., [Proc. SPIE 5314, 126-131 (2004)], Hitzenberger et al., [Opt Express. October 20; 11(20:2753-61 (2003)]). This technique includes the summing of the intensity signals in the 3D data set along one direction, for instance, along the axial direction of an Optical Coherence Tomography (OCT) scan, between two retinal tissue layers.

One common problem with this type of en-face image processing technique and other volume rendering techniques is the appearance of artifacts created by the involuntary motion of the subject's eye while a data set is being collected. The motion introduces relative displacements of the collected images so that salient physical features appear discontinuous in the resulting 3D data set, rendering the entire data set unreliable.

Another challenge that commonly occurs in the processing of OCT images is the central focus on reliable and reproducible layer segmentation in the B-scan (X-Z) images. Reliable layer segmentation can often be obtained when the retina is normal or with relatively small topographical changes. However, it becomes very unreliable, and in some cases impossible, to segment various layers accurately where there are significant layer profile alternations.

Therefore, there is a need for better processing and presentation of OCT image data.

### SUMMARY

In accordance with some embodiments of the present invention, a method of computer-aided diagnosis for ophthalmology includes acquiring an OCT dataset; obtaining an RPE fit from the OCT dataset; and generating a set of frontal en-face images based on the RPE fit, wherein the frontal en-face images are suitable for qualitative and quantitative assessment of a retina.

An OCT imaging system according to some embodiments includes an OCT imager that acquires OCT data; a computer coupled to the OCT imager, the computer executing instructions for: obtaining an RPE fit from the OCT dataset; and generating a set of frontal en-face images based on the RPE fit, wherein the frontal en-face images are suitable for qualitative and quantitative assessment of a retina.

These and other embodiments are further discussed below with reference to the following figures.

### BRIEF DESCRIPTION OF THE DRAWINGS

FIG. 1 shows an example of an OCT imager.

FIG. 2 shows a transformation function for image contrast enhancement according to some embodiments of the present invention.

FIG. 3 shows a diagram illustrating the X-Z longitudinal scan and X-Y transverse scan (C-scan).

FIGS. 4A and 4B show a classical C-scan (frontal en-face) view based on flat surfaces.

FIGS. 5A and 5B show a C-scan (frontal en-face) view based on the shape of retinal pigment epithelium (RPE) according to some embodiments of the present invention.

FIG. 6 is an example image of the RPE reference curve adapted to the RPE concavity.

FIG. 7 is an exemplary 4-up en-face display in accordance with some embodiments.

FIGS. 8A-8F show examples for pigment epithelium detachments (PED) intensity, texture, structure and morphology in Age-related Macular Degeneration (AMD) patients.

FIGS. 9A-9D show examples for PED intensity, texture, structure and morphology in PCV patients.

FIGS. 10A-10E show an example of region of interest (ROI) segmentation in some embodiments.

FIG. 11 shows an exemplary flowchart of the processing steps according to some embodiments.

### DETAILED DESCRIPTION

Optical Coherence Tomography (OCT) technology has been commonly used in the medical industry to obtain information-rich content in three-dimensional (3D) data sets. OCT can be used to provide imaging for catheter probes during surgery. In the dental- industry, OCT has been used to guide dental procedures. In the field of ophthalmology, OCT is capable of generating precise and high resolution 3D data sets that can be used to detect and monitor different eye diseases in the cornea and the retina. A new data presentation scheme and design, tailored to retrieve the most commonly used and expected information from these massive 3D data sets, can further expand the application of OCT technology for different clinical application and further enhance the quality and information-richness of 3D data set obtained by OCT technologies.

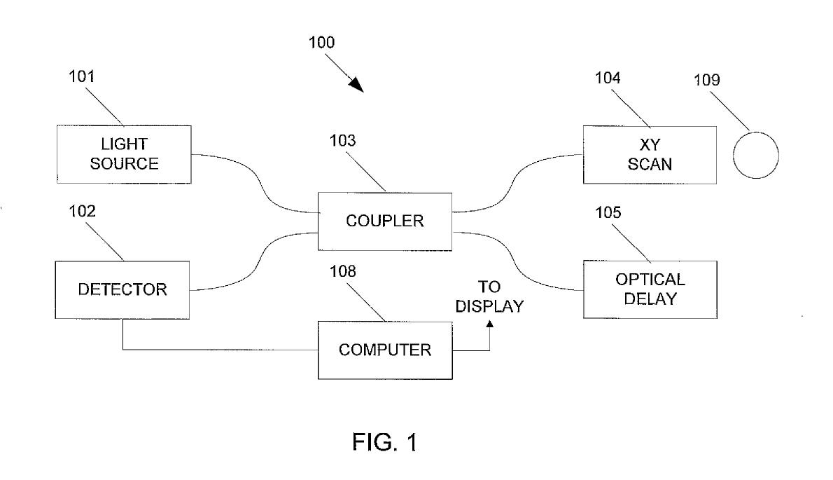

FIG. 1 illustrates an example of an OCT imager 100 that can be utilized in processing and presenting an OCT data set according to some embodiments of the present invention. OCT imager 100 includes light source 101 supplying light to coupler 103, which directs the light through the sampling arm to XY scan 104 and through the reference arm to optical delay 105. XY scan 104 scans the light across eye 109 and collects the reflected light from eye 109. Light reflected from eye 109 is captured in XY scan 104 and combined with light reflected from optical delay 105 in coupler 103 to generate an interference signal. The interference signal is coupled into detector 102. OCT imager 100 can be a time domain OCT imager, in which case depth (or A-scans) are obtained by scanning optical delay 105, or a Fourier domain imager, in which case detector 102 is a spectrometer that captures the interference signal as a function of wavelength. In either case, the OCT A-scans are captured by computer 108. Collections of A-scans taken along an XY pattern are utilized in computer 108 to generate 3-D OCT data sets. Computer 108 can also be utilized to process the 3-D OCT data sets into 2-D images according to some embodiments of the present invention. Computer 108 can be any device capable of processing data and may include any number of processors or microcontrollers with associated data storage such as memory or fixed storage media and supporting circuitry. In some embodiments, computer 108 can include a computer that collects and processes data from OCT 100 and a separate computer for further image processing. The separate computer may be physically separated.

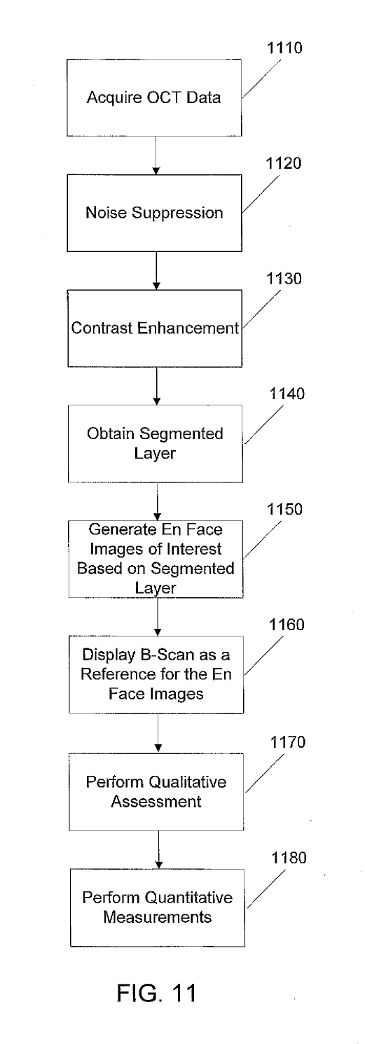

FIG. 11 shows an exemplary flowchart to obtain the qualitative assessment and quantitative measurements in some embodiments of the present invention. In step 1110, OCT data of interest can be acquired using an OCT imager 100. Then, a noise suppression process, step 1120, can be applied to reduce undesirable noise in the OCT data received in step 1110. In step 1130, contrast enhancement may be applied to the OCT data to enhance the contrast for future processing. In step 1140, a segmented layer of interest can be generated as a reference, using the enhanced OCT data from step 1130. For example, a retinal pigment epithelium (RPE) fit can be performed to obtain a fitted contour of the RPE. Other segmented layer of interest can include the inner limiting membrane (ILM) and the RPE. Using this RPE fit from step 1140, En Face images of interests can be generated in step 1150. In step 1160, a B-Scan display can further enhance the data presentation by providing a reference by displaying at least one B-Scan corresponding to the En Face images generated in step 1150. In step 1170, a qualitative assessment can be performed to provide qualitative assessment of the OCT data from step 1130. In some embodiments, quantitative measurements performed in step 1180 can also be obtained to provide objective and reproducible measurement capable for clinical diagnosis and evaluation.

### Noise Suppression and Contrast Enhancement

In some embodiments of the present invention, noise suppression can be used in the processing of OCT images in step 1120. One common approach is to apply linear or nonlinear spatial filters (e.g. window-averaging and median-filtering) to the images. One problem with this approach is that the parameters used in the spatial filters often need to be adjusted for images containing various levels of details (a balance between feature resolution and scale). It is not a trivial task to automatically adjust these parameters in general. Another simple but powerful approach to noise suppression is by temporal filtering such as frame averaging. This approach can substantially reduce the amount of noise by scanning multiple frames of the same region of interest (ROI) and then summing or averaging the repeated data. In many cases, however, eye movement may prevent application of this approach to obtain reasonable results. To alleviate this problem, image alignment methods based on the correlation among the acquired data can be used. An eye-tracking method and system can also be used to improve frame averaging. Moreover, using newer generations of FD-OCT technology with the increased scan speed of 70,000 to 100,000 A-scans per second may further assist in more accurate time averaging of multiple frames.

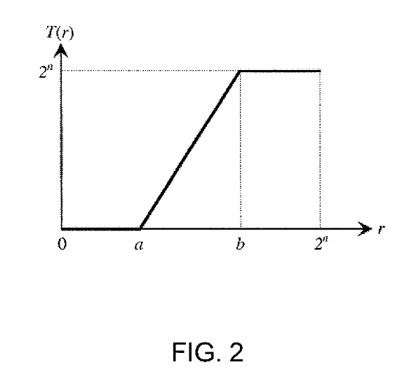

Contrast enhancement is another step in the processing of OCT images in some embodiments, and may be performed in step 1130. Contrast enhancement can accentuate features of interest and facilitate diagnosis of data in a desired intensity range. Contrast enhancement can be performed globally and locally. Global contrast enhancement uses transformation function such as a look up table (LUT). One of the simplest examples is contrast stretching; where a transformation function stretches a portion of the image histogram for amplitudes that contain desired information are placed across the whole amplitude range. FIG. 2 illustrates an example linear transformation function that takes values from the horizontal axis (r) and stretches value range from [a, b] to [0, 2n], where T(r) is the transformation function, a and b is the start and the end of the function, which is illustrated as a linear ramp in FIG. 2. Other functions may also be utilized.

In many cases, local contrast enhancement methods are more suitable in the analysis of OCT images and frontal en-face images. The image contents of these images inherently have a wide dynamic range of intensities. A classical solution to this problem is to use a local histogram equalization technique. Another commonly used local technique is spatial enhancement (sharpening) of high-frequency details in the ROI. An overview of similar techniques can be found in an article by D. H. Rao and P. P. Panduranga, “A survey on image enhancement techniques: classical spatial filter, neural network, cellular neural network, and fuzzy filter,” IEEE International Conference on Industrial Technology, pp. 2821-2826, December 2006.

### Frontal En-Face Views

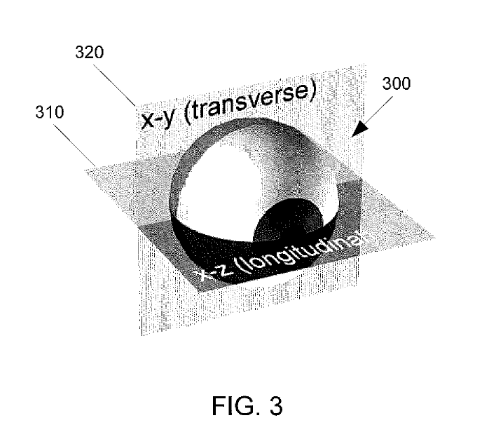

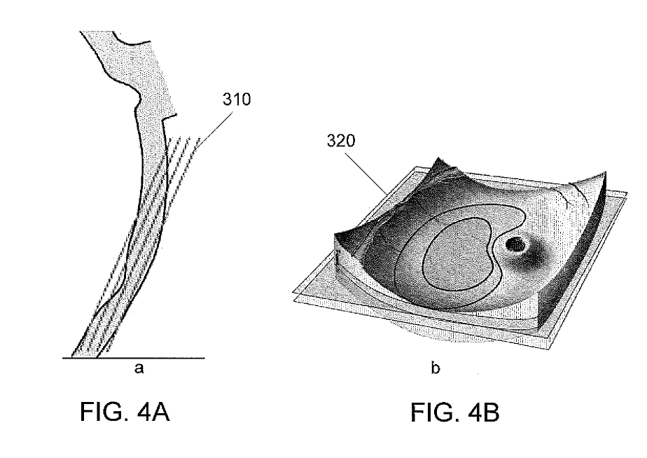

A Frontal En-face view is an observation direction along the axial direction of an OCT imager as in FIG. 1. FIG. 3 is an example pictorial representation of an eyeball 300 with commonly referenced image planes 310 and 320. An OCT B-scan is a 2D image along the longitudinal plane 310 that gives a X-Z view of the retina. A frontal en-face view or C-scan is a 2D image representation along the transverse direction, the X-Y plane 320. Cross-sectional images of these two views of the retina are shown in FIGS. 4A and 4B. A typical B-scan along longitudinal plane 310 in FIG. 4A and a typical C-scan along traverse plane 320 in FIG. 4B are simply flat illustrations cutting through the curved retina and do not conform to the curvature of a typical retina at the back of the eye.

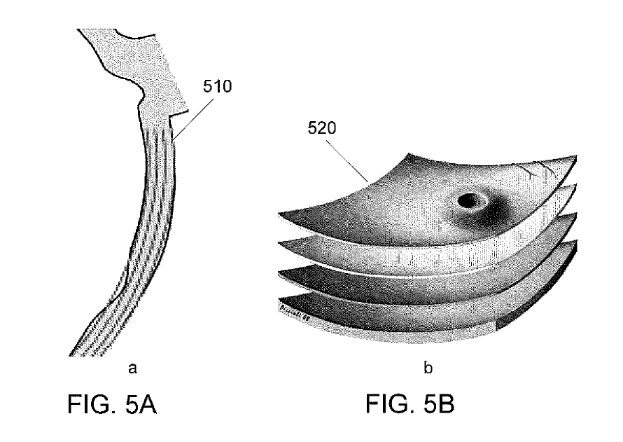

A more useful and clinically meaningful C-scan, as shown in FIGS. 5A and 5B, can be based on the general shape of the retinal pigment epithelium (RPE) or a fitted RPE curve or surface as a result of local smoothing or filtering of the RPE (RPE reference). Cross sectional images of the fitted longitudinal plane 510 and the fitted transverse plane 520 are shown in FIGS. 5A and 5B, respectively. In some embodiments, in step 1140 frontal en-face C scans following the general curvature of the RPE are employed to present OCT data that are more suitable for the diagnosis of retinal diseases. Such frontal en-face C scans only need to follow the general curvature of the retina and the precise layer segmentation of the RPE is not needed, as is commonly required in other applications. This approach alleviate the problem as shown in the cross sectional images in FIGS. 4A and 4B, while providing a more reliable and predictable OCT data image display without running into layer segmentation challenges such as disease retina, retina with complicated contour, and OCT data set with low quality due to poor signal to noise ratio or other imaging limitations. According to some embodiments, qualitative assessment and quantitative measurement can be provided to further enhance the clinical usefulness of navigating these information-intense 3D OCT data.

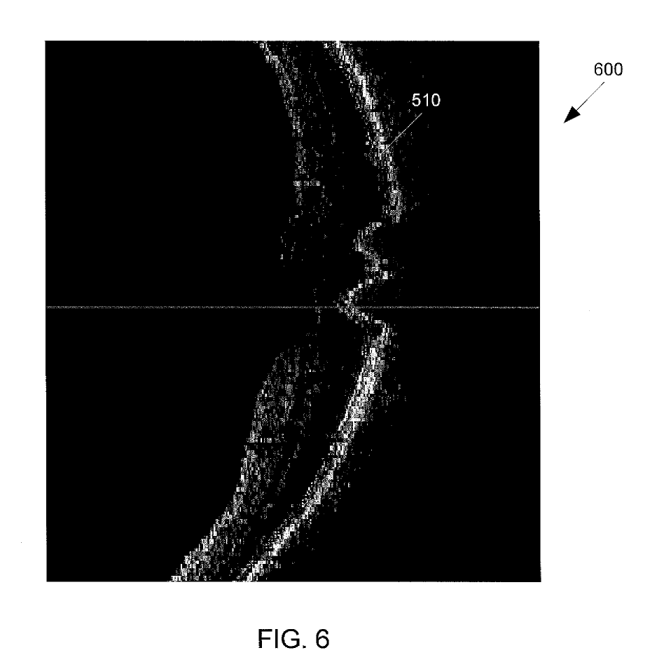

FIG. 6 is an example of a cross sectional OCT image 600 showing the fitted longitudinal plane in red 510. Varying the offsets and slice thickness in image 600 can reveal useful clinical information, such as RPE disruptions and irregularities. There are four areas of key interests to a clinician in order to determine the health of the retina during an eye exam, namely, 1) vitreo retinal interface abnormality, 2) edema, 3) drusen, geographic atrophy (GA), pigment epithelium detachments (PED), and 4) choroidal health. A data presentation scheme is disclosed to display information of key interests to the user in a reliable and systematic manner.

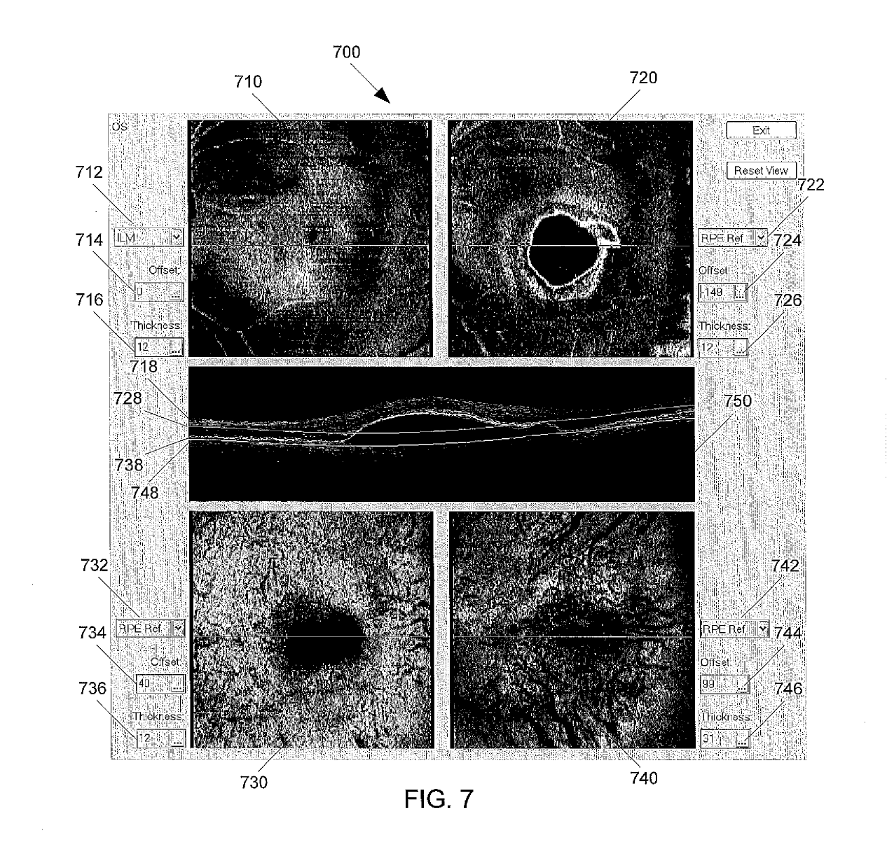

As discussed above, in step 1150 En Face Images are generated based on the RPE fit. FIG. 7 illustrates an exemplary 4-up frontal en-face display 700 of a sample PED to facilitate diagnosis of the above four retinal pathologies according to some embodiments of the present invention. In the exemplary display 700, 4 frontal en-face images are displayed to show information for 1) vitreo retinal interface abnormally 710, 2) edema 720, 3) drusen, GA, and PED 730, and 4) choroidal health 740, respectively. In step 1160, a cross-sectional image of a B-scan 750 can be displayed as a reference to show the relationship between images 710, 720, 730, and 740 and the cross-sectional spatial location of the OCT data set. In some embodiments, a color coded scheme is used to associate images 710-740 to the cross-sectional image 750. In FIG. 7, the contour 718 indicates the depth location of image 710; curve 728 associates with green-shaded image 720; curve 738 to image 730; and curve 748 to image 740. Typically, these curves and images utilize a color-coding or referencing scheme that can be used to show the relationship between images 710-740 and image 750.

To observe vitreo retinal interface abnormality, such as vitreous membrane detachment using image 710, an offset from the inner limiting membrane (ILM) can be applied, where the ILM is the boundary between the retina and the vitreous body. The ILM offset 712 can be set to −20 to 20 μm 714, with a slice thickness of 5 to 50 μm 716. In some embodiments, the ILM offset 714 is set to 0 μm and slice thickness 716 is set to 12 μm. To assess edema in the subject eye using image 720, the RPE reference offset 722 can be set to −300 to −20 μm 724, to −150 μm in some embodiments (i.e., 150 μm above RPE reference), with a slice thickness of 5 to 50 μm 726, to 12 μm in some embodiments, if the retinal full thickness is equal or less than 300 μm; in the alternative, the ILM reference offset can be set to 20 to 300 μm, to 160 μm in some embodiments (i.e., 160 μm below ILM), with a slice thickness of 5 to 50 μm, to 12 μm in some embodiments, if the retinal full thickness is more than 300 μm. To observe drusen, GA, PED and other retinal degeneration using image 730, the RPE reference offset 732 can be set to 10 to 100 μm 734, to 40 μm in some embodiments (i.e., 40 μm below RPE reference) with a slice thickness of 5 to 50 μm 736, to 12 μm in some embodiments. To observe characteristics of the choroid using image 740, the RPE reference offset 742 can be set to 50 to 350 μm 744 with a slice thickness of 5 to 50 μm 746; to 40 μm in some embodiments (i.e., 40 μm below RPE reference) with a slice thickness of 12 μm for thin atrophic choroid or to 100 μm (i.e., 100 μm below RPE reference) with a slice thickness of 30 μm for normal choroid. As discussed above, other segmented layer of interest, such as the ILM and the RPE, can be used for these assessments.

The discussed offsets and slice thicknesses are used to display these four key areas of interests; alternatively, a range of clinically meaningful values obvious to a person of ordinary skills in the art can be used in place. Additionally, the number of image displays can also be customized by the users based on their preferences so that different number of en face images of different number of key areas of interests can be displayed based on the specific workflow and evaluation of the user. The user interface can take in different customized inputs to allow different number of area of interests and to display a range of clinically meaningful values.

This presentation scheme can further highlight the morphological and structural characteristics of retinal edema such as Cystoid Macular Edema (CME) and choroidal vessels located at different depth, such as Sattler and Haller of the choroid.

### Qualitative Assessment

Images 710-740 in FIG. 7 are tailored to show the commonly evaluated conditions of the retina during an eye exam. As shown in step 1170, these high-resolution images provide qualitative assessment of various conditions of the subject eye. For example, these images can provide detailed information on different characteristic of these different retinal layers, such as intensity, texture, structure, and morphology. These characteristics are useful for the accuracy of diagnosis and the timeliness of needed treatments.

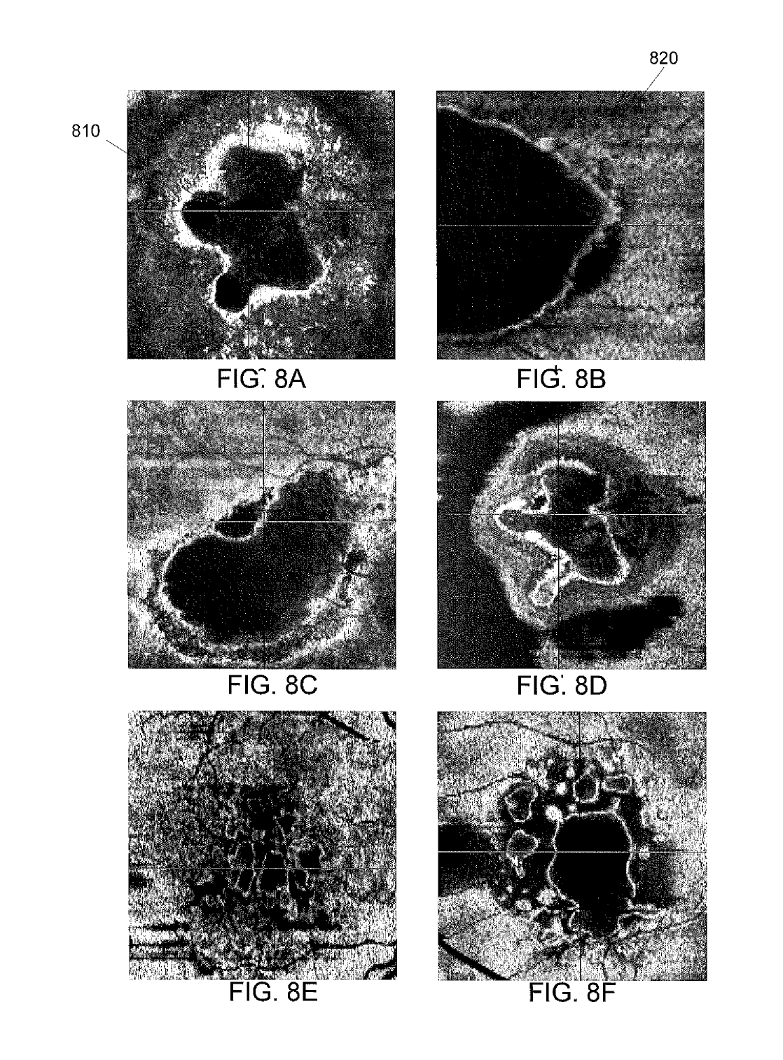

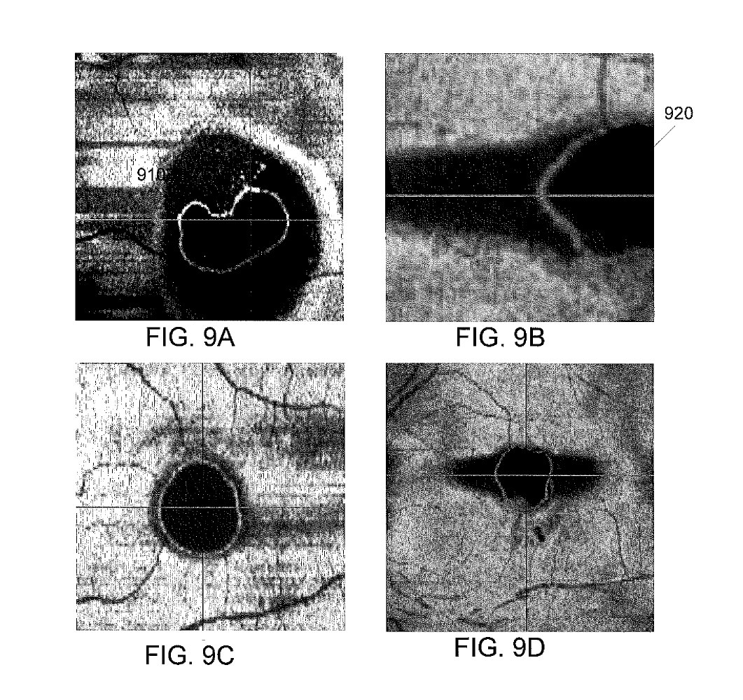

FIGS. 8A-8F and 9A-9D show examples of different forms of retinal diseases using these qualitative assessments. Using intensity assessment, one can evaluate the signal strength/intensity and homogeneity of the region of interest. Using texture assessment, one can evaluate the graininess of the region of interest. Structure assessment can show boundary thickness, smoothness and connectedness of the interested tissue and morphology assessment can be evaluated by the shape, size and regularity of the tissue.

FIGS. 8A-8F show example images of PED cases in Age-related Macular Degeneration (AMD) patients. In this pathology, the intensity of the central dark blob 810 is high and with non-homogenous signal strength (FIG. 8A). At the same time, the texture of the blob 810 is also coarse and grainy. In another example of this pathology, the structure of the dark blob 820 reveals that the boundary is non-smooth (jaggy), not well-connected, and its thickness is non-uniform (FIG. 8B). For morphology, distinctive features can be shown as qualitative assessment of this retinal pathology, such as irregular oval shape (FIG. 8C), multilobular blob (FIG. 8D), multi-cluster blobs (FIG. 8E), and multilobular plus clusters (FIG. 8F).

Another examples of the use of qualitative assessment can be appreciated in FIGS. 9A-9D, which shows images of PED cases in PCV patients. The intensity of the central blob 910 has low and homogenous strength (FIG. 9A). The texture of the blob 910 also shows little graininess. FIG. 9B shows the dark blob 920 has smooth boundary, well-connectedness and uniform thickness. For morphology, the central blob is predominantly circular in FIG. 9C and primarily oval in FIG. 9D. Neither of the blobs in FIGS. 9C and 9D is multilobular nor clustered.

### Quantitative Assessment

Qualitative assessment can provide useful information for clinical specialists for diagnosis and treatment, quantitative assessments can be further employed to provide objective, reproducible and accurate measurements to assist diagnosis and treatment.

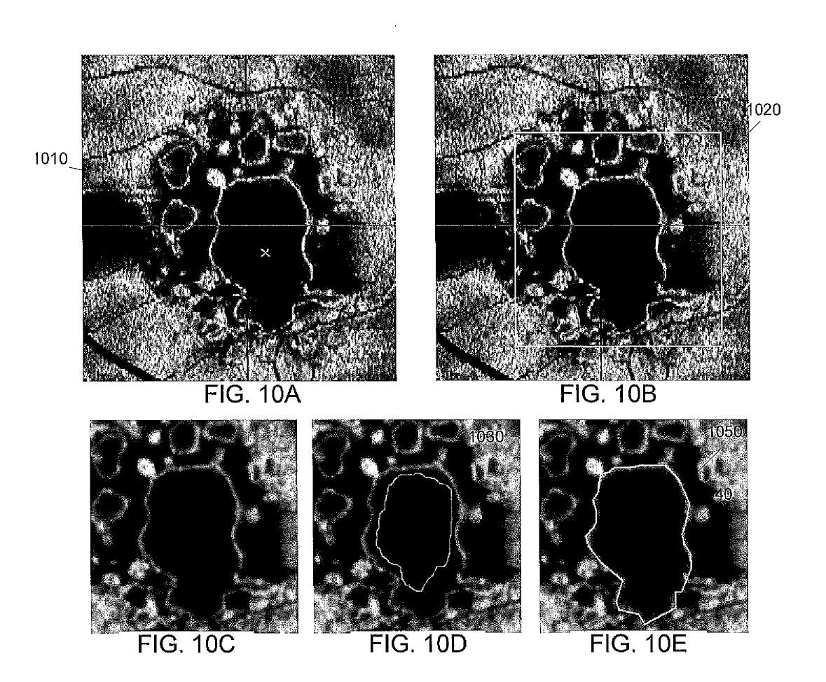

In step 1180, the first step to obtain quantitative measure is to identify the region of interest to be assessed. FIGS. 10A-10E illustrate a segmentation method to extract a region of interest. FIG. 10A shows an en-face image with the center dark blob 1010 as the region of interest. In some embodiments, the target region of interest 1010 has coordinates (x_{c}, y_{c}) as the centre of mass and the segmentation method uses an active contour model to identify the segmented region of interest (S) 1040, or its contour/border (∂S) 1050 as shown in FIG. 10E. Based on the coordinate (x_{c}, y_{c}) and the maximal allowable sizes of S 1040, a bounding box R 1020 containing S 1050 is automatically extracted (FIG. 10B). In this example, the region R 1020 is then multiplied with an inverse Gaussian function to suppress the heterogeneous image intensity inside R (FIG. 10C). Next, a preliminary blob region as shown in FIG. 10D is extracted from the background using a histogram threshold technique. The contour 1030 is used as the initial contour as an input to the active contour segmentation. An example of the final results of this segmentation technique of the blob region S 1040 and its contour/border 1050 are demonstrated in FIG. 10E.

After the region of interest is determined, quantitative measures of the characteristics discussed above can be parameterized, namely, intensity measures, texture measures, structure measures, and morphological measures.

### Intensity Measures:

The maximum, minimum, average, and standard deviation (homogeneity) of the intensity inside S are calculated and represented by Iₘₐₓ, Iₘᵢₙ, I_{avg}, and I_{std}, respectively.

### Texture Measures:

The texture measure is defined by the ratio of edge (grainy) pixels inside S to the total number of pixels in S. It can be explicitly represented by

mₜₓ=(Area[edge pixels inside S])/(Area[S]),

where Area[S] denotes the pixel number of S. The edge pixels can be detected by using the Canny edge operator for an example.

### Structure Measures:

The smoothness, connectedness, and thickness uniformity of the blob border curve as are computed by

mₛₘ=1.0/(average of the curvature change along ∂S),

m_{cn}=1.0/(standard deviation of the edge strength along ∂S),

mₜᵤ=1.0/(standard deviation of the edge thickness along ∂S),

respectively. If as is smooth, the curvature change along as becomes small in average, and hence the smoothness measure, mₛₘ, would be large. The edge strength of an edge pixel is computed by its edge slope along ∂S. If ∂S is well-connected, the edge strength along ∂S would have small variations, and hence the connectedness measure, m_{cn}, would become large. Similarly, if ∂S has uniform thickness, the standard deviation of the edge thickness would be small, and hence the thickness uniformity measure, mₜᵤ, would become large.

### Morphological Measures:

Pattern spectrum, a shape-size descriptor, can be used to quantitatively evaluate the shape and size of S. Large impulses in the pattern spectrum at a certain scale indicate the existence of major (protruding or intruding) substructures of S at that scale. The bandwidth of the pattern spectrum, m_{bw}, can then be used to characterize the size of S. An entropy-like shape-size complexity measure based on the pattern spectrum, m_{ir,} can be used to characterize the shape and irregularity of S. Mathematically, the pattern spectrum of S relative to a binary structuring element B (disk shape) of size (scale) r, is denoted by PS_{S}(r, B). The measures m_{bw} and mᵢᵣ are defined by

m_{bw}=rₘₐₓ−rₘᵢₙ, and

mᵢᵣ=−Σp(r)log [p(r)],

respectively. The scale parameters rₘₐₓ and rₘᵢₙ denote the maximum and minimum size in PS_{S}(r, B), respectively. Here p(r)=PS_{S}(r, B)/Area(S) is the probability function by treating PS_{S}(r, B) from a probabilistic viewpoint. The maximum value of mᵢᵣ is attained whenever the pattern spectrum is flat, indicating that S is very irregular or complex by containing B (disk) patterns of various sizes. Its minimum value (0) is attained whenever the pattern spectrum contains just an impulse at, say, r=k; then S is simply a pattern B (disk) of size k and therefore considered to be the most regular (or the least irregular).

It should be appreciated that alternative and modifications apparent to one of ordinary skills in the art can be applied within the scope of the present inventions. For example, the offset value, slice thickness in the 4-up en-face representation, and the quantitative measures can be varied from the specific embodiments disclosed herein within the scope and spirit of the subject invention.

We claim:

1. A method of computer-aided diagnosis for ophthalmology, comprising:
acquiring an OCT dataset;
obtaining a segmented layer of interest from the OCT dataset;
generating a set of frontal en-face images based on the segmented layer of interest; and
displaying the set of frontal en-face images,
wherein the frontal en-face images are suitable for qualitative and quantitative assessment of a retina.

2. The method of claim 1, further including processing the OCT dataset for noise suppression.

3. The method of claim 1, further including processing the OCT dataset for contrast enhancement.

4. The method of claim 1, wherein obtaining a segmented layer of interest includes determining an RPE fit, an ILM layer, or an RPE layer.

5. The method of claim 1, wherein the qualitative assessment includes structural and morphological assessment on at least one area of interests.

6. The method of claim 5, wherein the structural assessment includes computation of metrics, including at least one of a set of metrics consisting of intensity, homogeneity, boundary thickness, smoothness, connectedness of the area of interest.

7. The method of claim 5, wherein the morphological assessment includes computation of metrics, including one of shape, size, and regularity of the area of interest.

8. The method of claim 7, wherein the area of interest includes a retina, a choroid, an interface of vitreous-retina, a retina-choroid, and a choroid-sclera.

9. The method of claim 7, wherein the morphological assessment includes examination of the shape and dimensions of retina and choroid, as well as the interfaces of vitreous-retina, retina-choroid, and choroid-sclera.

10. The method of claim 5, wherein RPE structure and morphology provide for early detection of macular diseases.

11. The method of claim 10, wherein macular diseases includes drusen, geographic atrophy, and pigment epithelium detachments.

12. The method of claim 8, wherein choroidal vascular changes provide detection of choroidal melanomas.

13. The method of claim 8, wherein choroidal layer thickness and volume provide detection of choroidal neovascularization and age related macular degeneration.

14. The method of claim 1, wherein the set of en-face images includes a plurality of images based on the segmented layer of interest and a B-scan image and displaying the set of en-face images includes simultaneously displaying the set of en-face images on a single display.

15. The method of claim 14, wherein the set of en-face images includes a vitreo reintal interface image, an edema image, a retinal degeneration image, a choroidal image, and a cross-sectional image of a B-scan.

16. An OCT imaging system, comprising:
an OCT imager that acquires OCT data;
a computer coupled to the OCT imager and a display, the computer executing instructions for:
obtaining an RPE fit from the OCT dataset;
generating a set of frontal en-face images based on the RPE fit; and
displaying the set of frontal en-face images
wherein the frontal en-face images are suitable for qualitative and quantitative assessment of a retina.

17. The system of claim 16, further including processing the OCT dataset for noise suppression.

18. The system of claim 16, further including processing the OCT dataset for contrast enhancement.

19. The system of claim 16, wherein obtaining an RPE fit from the OCT database includes determining the curvature of the RPE.

20. The method of claim 16, wherein the set of en-face images includes a plurality of images based on the segmented layer of interest and a B-scan image and displaying the set of en-face images includes simultaneously displaying the set of en-face images on the display.

21. The method of claim 20, wherein the set of en-face images includes a vitreo reintal interface image, an edema image, a retinal degeneration image, a choroidal image, and a cross-sectional image of a B-scan.
